(19) United States
(12) Patent Application Publication
(10) Pub. No.: US 2026/0213023 A1
(43) Pub. Date: Jul. 23, 2026

(54) SELECTIVELY REPURPOSING CONSUMER DEVICE DATA

(71) Applicant: KONINKLIJKE PHILIPS N.V., EINDHOVEN (NL)

(72) Inventors: EDUARD GERARD MARIE PELSSERS, PANNINGEN (NL); NEIL FRANCIS JOYE, WAALRE (NL); HANS WILLEM VAN KESTEREN, EINDHOVEN (NL); JOZEF HUBERTUS GELISSEN, HERTEN (NL); OTTO PAULUS HENRIKUS KOKKE, EINDHOVEN (NL)

(21) Appl. No.: 19/141,113

(22) PCT Filed: Dec. 14, 2023
(86) PCT No.: PCT/EP2023/085844

§ 371 Date: Jun. 19, 2025

Related U.S. Application Data
(60) Provisional application No. 63/433,509, filed on Dec. 19, 2022.

(51) Int. Cl. G16H 50/70; G16H 50/20

(52) U.S. Cl. CPC G16H 50/70; G16H 50/20

(57) ABSTRACT

A system for deriving medical characteristics from data includes a gateway computer comprising a memory that stores instructions and a processor that executes the instructions. The instructions cause the gateway computer to: receive first data from a first consumer device; filter the first data for quality based on a first pattern of the first data and to eliminate artefacts to produce first filtered data; derive, from the first filtered data, a first physiological parameter; receive, from a second consumer device, second data; filter the second data for quality based on a second pattern of the second data and to eliminate artefacts to produce second filtered data; and derive, from the second filtered data, a second physiological parameter.

### BACKGROUND

In medical fields, verification and validation of diagnostic medical data is important since the resultant diagnosis may be the basis of medical care. The highest standards are used to ensure that medical data is valid. In some jurisdictions, medical data is strictly regulated, such as by the Medical Device Regulation (MDR) and In-Vitro Device Regulation (IVDR). The MDR covers medical devices that measure inside the human body and the IVDR covers medical devices that measure a component of a biofluid outside the human body.

In recent years, consumer devices have been developed that can measure physiological parameters such as heart rate, respiration, and energy expenditure. In consumer devices incorporating a photoplethysmography (PPG) sensor, blood parameters can be assessed in a non-invasive way. PPG is an optical technology to detect blood volume changes in the underlying tissue. A conventional PPG sensor monitors the oxygen saturation (SpO2) of the arterial blood in the tissue while the pulsation of the PPG signal reflects the heart rate. Although these consumer devices are less strictly regulated than medical devices, that does not mean that the information provided by these consumer devices is not valuable. Information provided by consumer devices may be useful for co-determining the condition of a person, for determining measures to prevent illness, and even for support in establishing a healing plan. The distinguishing features of these consumer devices is that they can gather (i) data reflective of physiological conditions well before the person is ill and also during illness, (ii) under various conditions such as at rest, while doing exercising and while sleeping and (iii) twenty four hours per day and seven days per week for years. The benefit of having access to such massive data even before a person is ill is incalculable, but strict measures must be put in place before use of such data complies with standards such as the MDR and IVDR.

Since data from consumer devices may be gathered in a relatively uncontrolled environment outside a medical facility, the data is currently not allowed to serve as a basis for medical handling. Various sources of decreased accuracy from such data include artefacts. Artefacts may be due to incorrect use of the consumer device such as at a non-specified body area, in a non-calibrated state, or by more than one person. Artefacts may also result from application of a consumer device outside the intended use such as outside of intended ranges in humidity or temperature, by persons outside of an intended age range, or by use by more than one person who does not declare their identification to the consumer device. However, the data gathered by these consumer devices does have value in areas of prevention and therapy.

Accordingly, solutions are required for using data from consumer devices without violating the MDR or IVDR. Selective repurposing of consumer device data is called for, so long as technological mechanisms are put in place to ensure compliance with standards such as the MDR and the IVDR.

### SUMMARY

According to an aspect of the present disclosure, a system for deriving medical characteristics from data includes a gateway computer. The gateway computer includes a memory that stores instructions and a processor that executes the instructions. When executed by the processor, the instructions cause the gateway computer to: receive first data from a first consumer device; filter the first data for quality based on a first pattern of the first data and to eliminate artefacts to produce first filtered data; derive, from the first filtered data, a first physiological parameter; receive, from a second consumer device, second data; filter the second data for quality based on a second pattern of the second data and to eliminate artefacts to produce second filtered data; and derive, from the second filtered data, a second physiological parameter.

According to another aspect of the present disclosure, a method for deriving medical characteristics from data includes receiving, at a gateway computer comprising a memory that stores instructions and a processor that executes the instructions, from a first consumer device, first data; filtering, by the gateway computer, the first data for quality based on a first pattern of the first data and to eliminate artefacts to produce first filtered data; deriving, by the gateway computer, from the first filtered data, a first physiological parameter; receiving, by the gateway computer, from a second consumer device, second data; filtering, by the gateway computer, the second data for quality based on a second pattern of the second data and to eliminate artefacts to produce second filtered data; and deriving, by the gateway computer, from the second filtered data, a second physiological parameter.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1 illustrates a system for selectively repurposing consumer device data, in accordance with a representative embodiment.

FIG. 2 illustrates a method for selectively repurposing consumer device data, in accordance with a representative embodiment.

FIG. 3A illustrates another method for selectively repurposing consumer device data, in accordance with a representative embodiment.

FIG. 3B illustrates another method for selectively repurposing consumer device data, in accordance with a representative embodiment.

FIG. 4 illustrates another method for selectively repurposing consumer device data, in accordance with a representative embodiment.

FIG. 5 illustrates an example of variable interbeat time duration for selectively repurposing consumer device data, in accordance with a representative embodiment.

FIG. 6 illustrates average pattern factor per time segment as a function of the number of segments measured in a chronologic sequence for selectively repurposing consumer device data, in accordance with a representative embodiment.

FIG. 7 illustrates a computer system, on which a method for selectively repurposing consumer device data is implemented, in accordance with another representative embodiment.

### DETAILED DESCRIPTION

In the following detailed description, for the purposes of explanation and not limitation, representative embodiments disclosing specific details are set forth in order to provide a thorough understanding of embodiments according to the present teachings. However, other embodiments consistent with the present disclosure that depart from specific details disclosed herein remain within the scope of the appended claims. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. Definitions and explanations for terms herein are in addition to the technical and scientific meanings of the terms as commonly understood and accepted in the technical field of the present teachings.

It will be understood that, although the terms first, second, third etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

As used in the specification and appended claims, the singular forms of terms ‘a’, ‘an’ and ‘the’ are intended to include both singular and plural forms, unless the context clearly dictates otherwise. Additionally, the terms “comprises”, and/or “comprising,” and/or similar terms when used in this specification, specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to”, “coupled to”, or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

The present disclosure, through one or more of its various aspects, embodiments and/or specific features or sub-components, is thus intended to bring out one or more of the advantages as specifically noted below.

As described herein, data from consumer devices which belong to consumers may be combined with data from medical devices which may or may not be owned by consumers. The data from consumer devices will be subject to checks, as part of the teachings herein, before use to ensure compliance with government standards for data protection. Once compliance is ensured, data reflective of medical information for the consumers may be combined with data from medical devices and subject to analysis and storage on behalf of the consumers. The values of physiological parameters derived from consumer devices may be combined with conventional values to determine an illness or diagnosis, and this may result in early diagnosis of oncoming illnesses using mechanisms that may be customized for each individual. In medical fields, the innovations described herein may be used to detect deterioration, to provide support in the healing process, and to improve quality of life after hospitalization by providing accurate measuring modalities and systems that process the data from consumer devices.

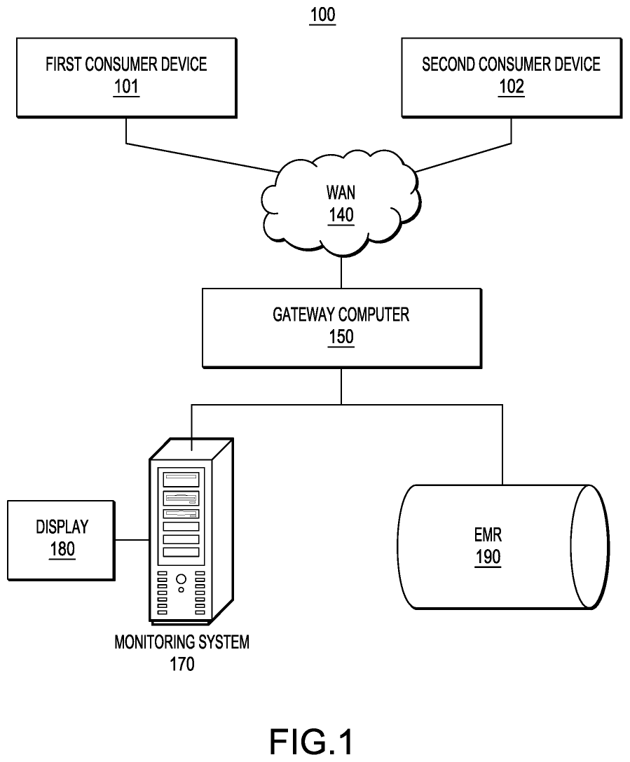

FIG. 1 illustrates a system 100 for selectively repurposing consumer device data, in accordance with a representative embodiment.

The system 100 in FIG. 1 is a system for selectively repurposing consumer device data and includes components that are distributed. The system 100 includes a first consumer device 101, a second consumer device 102, a wide area network 140, a gateway computer 150, a monitoring system 170, a display 180 and an EMR 190. The system 100 classifies data from the first consumer device 101 and the second consumer device 102 to ensure appropriate use of the data when provided for medical uses.

The first consumer device 101 and the second consumer device 102 are representative of a plurality of consumer devices, including dozens, hundreds, thousands, or millions of consumer devices. While for one person the number of consumer devices utilized may be limited, typically between one and ten, many persons may use a total of millions of devices that can connect to the gateway computer 150 via the wide area network 140. The first consumer device 101 and/or the second consumer device 102 may comprise a consumer cell phone including consumer smart phones, a networked consumer watch including a networked smart watch, a networked consumer scale, a networked consumer computer, a networked consumer tablet, a networked vehicle such as a networked consumer automobile, motorcycle or boat, a networked consumer television including a consumer smart television, and other types of networked electronic devices owned by consumers and which are capable of directly sensing or otherwise deriving or usable to assist in deriving certain physiological data and/or biomarker data. The first consumer device 101 and the second consumer device 102 may be used when a person is healthy or ill, and for some consumer devices, in various states such as at rest, doing exercise and asleep. Further the data from the first consumer device 101 and/or the second consumer device 102 may be collected twenty four hours per day and seven days per week for years in typical intervals of seconds to days and depending on the specific gathered data in intervals of a minute to a day or even in intervals of thirty minutes to several hours.

The wide area network 140 is representative of the Internet.

The gateway computer 150 may comprise a computer such as a server which receives data at an internet protocol address. The gateway computer 150 may receive data via one or more websites, or data directly addressed to the internet protocol address. The gateway computer 150 receives the data over the wide area network 140 from the first consumer device 101 and the second consumer device 102, and potentially from dozens, hundreds, thousands or millions of other consumer devices. The gateway computer 150 is representative of a plurality of gateway computers, such as when the gateway computer 150 and the monitoring system 170 are provided separately for separate entities.

The gateway computer 150 may be provided in the cloud, such as in a data center. The gateway computer 150 performs tasks such as authentication a user of the first consumer device 101 and the second consumer device 102, cleaning data containing or corresponding to excessive artefacts, pattern detection for the cleaned data, and detection of deterioration in the health of the user. The gateway computer 150 may release data to the monitoring system 170 and the EMR 190 after processing.

The monitoring system 170 may comprise one or more computers and other types of electronic equipment, such as a patient monitor, and is configured to monitor subjects corresponding to the first consumer device 101, the second consumer device 102 and other consumer devices. The monitoring system 170 may be provided by an entity such as medical facility or as a third-party service on behalf of multiple entities which provide medical services to consumers.

The display 180 is representative of a display used by users at the monitoring system. Users at the monitoring system 170 may include medical professionals responsible for monitoring health of consumers using the first consumer device 101 and the second consumer device 102. The display 180 may be local to the monitoring system 170 or may be remotely connected to the monitoring system 170. The display 180 may be connected to the monitoring system 170 via a local wired interface such as an Ethernet cable or via a local wireless interface such as a Wi-Fi connection. The display 180 may be interfaced with other user input devices by which users can input instructions, including mouses, keyboards, thumbwheels and so on. The display 180 may be a monitor such as a computer monitor, part of a patient monitor, a display on a mobile device, an augmented reality display, a television, an electronic whiteboard, or another screen configured to display electronic imagery. The display 180 may also include one or more input interface(s) such as those noted above that may connect to other elements or components, as well as an interactive touch screen configured to display prompts to users and collect touch input from users.

The EMR 190 is representative of a large memory system for electronic medical records. The EMR 190 may be provided with the monitoring system 170 or separately from the monitoring system. The EMR 190 may be provided for an office, a facility with multiple offices, or an entity with multiple facilities each with one or more office.

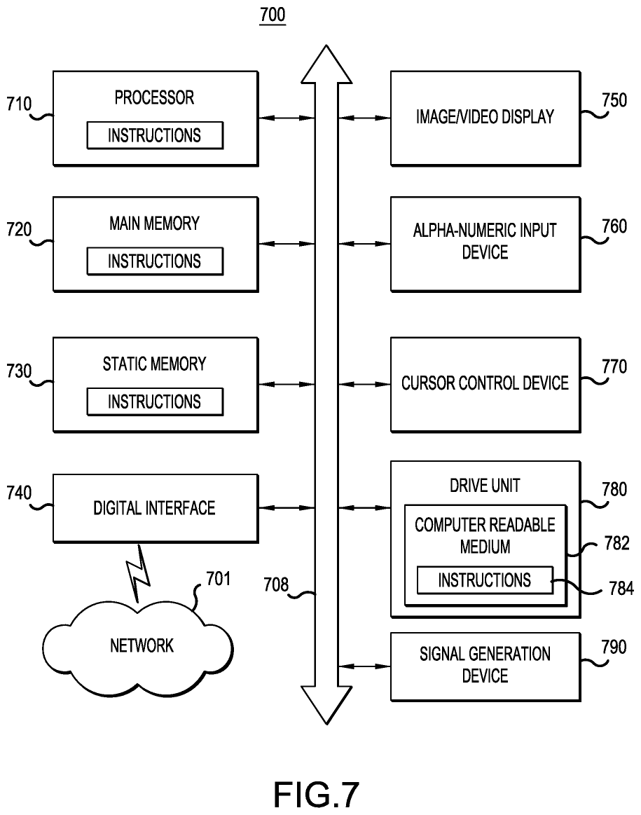

Any of the first consumer device 101, the second consumer device 102, the gateway computer 150 or the monitoring system 170 may include a controller. Multiple different elements of the system 100 in FIG. 1 may include a controller. A controller includes at least a memory that stores instructions and a processor that executes the instructions. A computer that can be used to some instances of the first consumer device 101 and/or the second consumer device 102 as well as the gateway computer 150 and the monitoring system 170 is depicted in FIG. 7, though a controller may include more or fewer elements than depicted in FIG. 7. Any controller may include interfaces, such as a first interface, a second interface, a third interface, and a fourth interface. One or more of the interfaces may include ports, disk drives, wireless antennas, or other types of receiver circuitry that connect the controller to other electronic elements. One or more of the interfaces may also include user interfaces such as buttons, keys, a mouse, a microphone, a speaker, a display separate from the display 180, or other elements that users can use to interact with the controller such as to enter instructions and receive output.

A controller of the first consumer device 101, the second consumer device 102, the gateway computer 150 or the monitoring system 170 may perform some of the operations described herein directly and may implement other operations described herein indirectly. For example, a controller of the gateway computer 150 may directly receive and process data from the first consumer device 101 and the second consumer device, and then indirectly control other operations by sending results for additional processing at the monitoring system 170, for display on the display 180, and/or for storage in the EMR 190. Thus, the controller of the gateway computer 150 may indirectly control other operations performed directly by other elements in FIG. 1. Accordingly, the processes implemented by the controller of the gateway computer 150 when a processor executes instructions from the memory may include steps not directly performed by the controller of the gateway computer 150.

Some or all communications between the nested application on a consumer device and the gateway computer 150 may be encrypted. In some embodiments, the consumer devices and the gateway computer 150 may engage in a required handshake before interacting.

Natural processes follow particular patterns. When a specific disorder occurs, specific patterns are disturbed. Recognition of these patterns can be used to distinguish healthy individuals, such as when a pattern changes due to an oncoming disorder. Artefacts should be identified and filtered out if data from a consumer device is to be trusted. The patterns of data from consumer devices such as the first consumer device 101 and the second consumer device 102 may be used to distinguish data from healthy persons to data from persons with oncoming disorders. While artefacts may induce a form of randomness, the data from healthy persons tends to have a form of constant evolvement and the data from a person with an oncoming disorder tends to have a form of trend to another state of the pattern.

Artefacts to be filtered at the consumer devices and the gateway computer 150 may be classified into avoidable artefacts that can be recognized by the system 100 a-priori before measurements and unavoidable artefacts that will influence the measurement a-priori with unknowing effect. An example of an avoidable artefact is when a person does not declare his/her identity to the first consumer device 101 or the second consumer device 102. Unavoidable artefacts may comprise (i) motion artefacts that are introduced by movement of the person during sleep, (ii) incorrect use of the consumer device, such as when using the consumer device on a body area that is not included in the intended use, (iii) wrong fixation on a body area such as when a wrist watch with measuring capability is too loosely placed on the wrist, (iv) when a device is used in an area where EMI radiation is too high, or the humidity is too high, or the temperature is too high or too low.

Some unavoidable artefacts may transition partially or fully to avoidable artefacts. For instance, motion artefacts can be detected by an accelerometer, and data may be excluded when the acceleration is too high. As another example, a consumer device may identify the location of the body area by probing properties of the skin that are different from location to location. Wrong fixation on the body can be detected by identifying the force that a strap is closed. Environmental properties such as temperature and humidity may be recorded by the consumer device and checked to ensure the consumer device is being used as intended. Some unavoidable artefacts will remain as unavoidable and will require filtering at the consumer devices or at the gateway computer 150. The filtering will alleviate any need for additional measurements to identify the artefacts and will enable continuous or semi-continuous measurement without being interrupted by suddenly occurring artefacts.

As set forth above, the system 100 is for deriving medical characteristics from data. The system 100 includes the gateway computer 150, and the gateway computer 150 includes at least a memory that stores instructions and a processor that executes the instructions. When executed by the processor, the instructions cause the gateway computer 150 to: receive first data from a first consumer device 101; filter the first data for quality based on a first pattern of the first data and to eliminate artefacts to produce first filtered data; derive, from the first filtered data, a first physiological parameter; receive, from a second consumer device 102, second data; filter the second data for quality based on a second pattern of the second data and to eliminate artefacts to produce second filtered data; and derive, from the second filtered data, a second physiological parameter.

As an example, chemical biomarkers such as the level of oxygen binding to hemoglobin in blood may be measured based on data from the first consumer device 101 and the second consumer device 102. By providing access to large amounts of data from consumer devices even before a person is ill, the ability to diagnosis, treat and even prevent some illnesses may be enhanced using the system 100. However, since the data from these consumer devices may be gathered in a relatively uncontrolled environment outside a medical facility, value of the data from these consumer devices must be evaluated before use for medical purposes every time such data is measured. The system 100 allows gathering and classifying of the data from these consumer devices, and the gateway computer 150 filters the data with respect to a quality level utilizing comparable data obtained by medical devices.

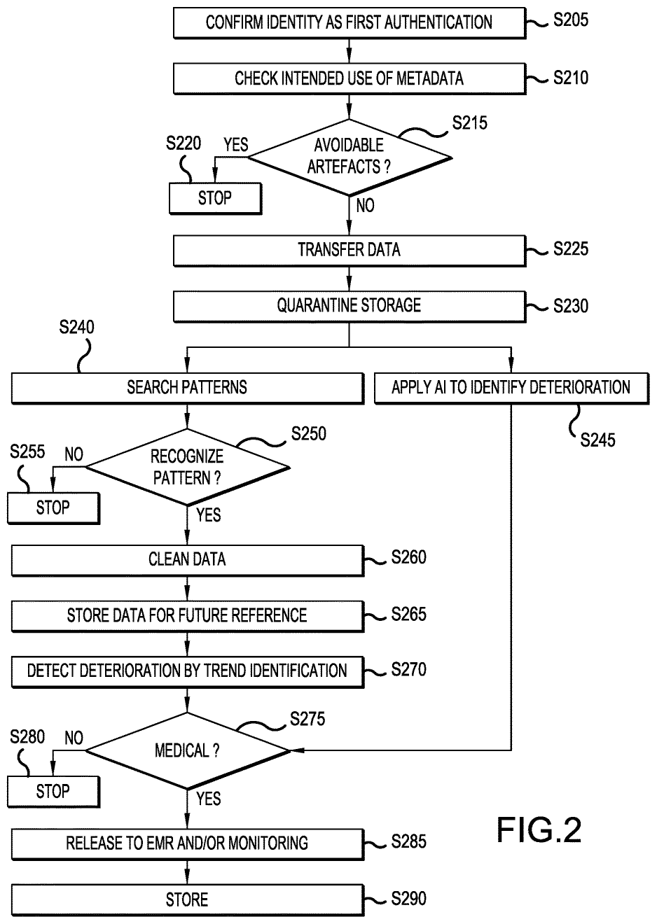

FIG. 2 illustrates a method for selectively repurposing consumer device data, in accordance with a representative embodiment.

The method of FIG. 2 may be performed by a back-end system such as a system that includes the gateway computer 150, the monitoring system 170, the display 180 and the EMR 190 in FIG. 1.

Before the method of FIG. 2 is performed, an application may be nested into a consumer device such as the first consumer device 101 and/or the second consumer device 102. The nested application(s) may be installed on the consumer devices at manufacture, or otherwise downloaded from an application store or installed as a software update. The nested application collects data and metadata that characterizes context for measured parameters in a particular environment which should be within the intended use of the nested application. For example, the nested application may collect acceleration data that can be linked to motion artefacts.

At S205, the method of FIG. 2 begins with confirming an identity as a first authentication. The nested application(s) may collect data and metadata that characterizes particular avoidable artefacts. For example, the nested application(s) may collect data and metadata reflecting that a person does not declare his identity to the consumer device as the first authentication, in which case the nested application may reject further use of the collected data and metadata and will not send the data to the gateway.

At S210, the intended use of the data received by or sensed by the consumer device is checked by analysis of metadata. For instance, in case that an analysis is only valid for persons with a certain age, for instance adults, the data may be declared invalid when a child is using the device.

At S215, a check is made for avoidable artefacts. An avoidable artefact may be the lack of a voluntary confirmation of identity by the user, as set forth above for S205. Another avoidable artefact that may result in the nested application stopping the collection of data and metadata is the drift of a signal due to lack of calibration. The nested application may check the time stamp of the last calibration and, if incorrect, will block sending data to the gateway computer 150. Yet another avoidable artefact is when a person provides incorrect identification information, such as a wrong age, gender or health status to the measuring consumer device. Before the nested application will send parameter data to the gateway computer 150, the nested application may initiate a request according to a standardized communication protocol to the gateway computer. A request may include identification information of a person for which data is being searched. The gateway computer 150 may connect to the EMR 190 or another storage system which stores the identity of the person. Authentication will occur by comparing the identification information with the identification as declared by the consumer device with the nested application. If the declared identification is correct, the nested application may send the parameter data to the gateway computer 150. Each time data is collected by the consumer device, the nested application may check if the person has been identified by the consumer device, for instance by fingerprints, a password and/or another identification method.

If the metadata includes avoidable artefacts (S215=Yes), the method of FIG. 2 stops at S220.

If the metadata does not include avoidable artefacts (S215=No), at S225 the method of FIG. 2 includes transferring data. The transfer at S225 may be according to a standardized communication protocol between the nested application and the gateway computer 150.

In some embodiments, formats for data acceptable to the gateway computer 150 may be defined. For example, insofar as a gateway computer 150 is expected to receive voluminous data from numerous consumer devices of a wide variety, the provider of the gateway computer 150 may mandate that packets sent to the gateway computer 150 comply with a specific format so that fields in the packets can be quickly and efficiently processed. The required format may include specific fields in particular locations relative to the start of an internet protocol packet, such as a source identification field to identify the first consumer device or the second consumer device and a source identification field to identify the subject.

At S230, the data is quarantined in storage. The gateway computer 150 may quarantine parameter data received from the nested application on the first consumer device 101 or the second consumer device 102 in order to verify the data before releasing the data for use.

At S245, after S230, artificial intelligence is applied to identify deterioration based on the data. The artificial intelligence is applied via an artificial intelligence model and in parallel with a separate subprocess between S240 and S270. Artificial intelligence applied at S245 to identify deterioration is a first mechanism for identifying deterioration, whereas trend identification applied at S270 is a second mechanism for identifying deterioration. An artificial intelligence engine may be applied at the gateway computer 150 at S245 to identify deterioration. By virtue of the large amounts of data available from consumer devices, controlled experiments may be performed with a-priori healthy persons and ill persons to create a training set. A trained artificial intelligence model applied at S245 is able to distinguish patterns of healthy and ill persons. The pattern recognition algorithm as used for artefact detection may be based on a trained artificial intelligence model

The change in patterns described with respect to S270 may also be learned by an artificial intelligence engine when training data includes data for persons who change from healthy to ill. The artificial intelligence engine is a tool for the system 100 to identify if a person is healthy or ill.

At S240, also after S230, a search is made for patterns in the stored data. The cloud gateway may perform the search at S240 as an internal authentication. The quarantined data may be compared with previous data transfer sessions from the same user. Although the data patterns can change over time, particular fingerprints of the pattern should not. The search for conformity with existing data patterns may represent an independent authentication of the data to confirm the data belongs to the intended person. If yes, the data will be released for the second step.

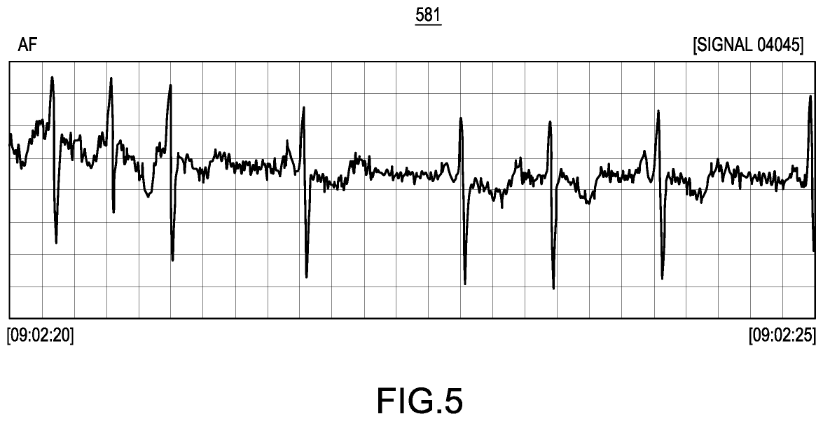

In some embodiments, biometrics may be used for the patterns searched at S240. Biometrics may include, for example, fingerprint or eye-iris images, or heart rate from a PPG sensor. For example, a time between heart beats may be checked as a pattern factor, and compared as a function of time with previous heartbeat measurements. FIG. 5 shows an example of variable interbeat time duration used for an internal check to authenticate data at the gateway computer 150 at S240.

At S250, after S240, a determination is made whether a pattern is recognized in the stored data which is searched at S240. The pattern recognition may serve as a second authentication at the gateway computer 150. The pattern recognition is used to identify real signals from artefacts based on particular random-like deviations in the pattern. Data sets with too much data caused by artefacts may be filtered out.

In well controlled experiments, first data is gathered without artefacts and second data is gathered with induced and controlled artefacts. The impact of the artefacts on the measured parameters is quantified using a pattern factor. The pattern factor may be determined by measuring the peak to peak time-distance in a relatively short time-segment, for instance containing 100 peaks. The average of the peak-to-peak time-distance may be defined as the pattern factor. Subsequently the variation in the pattern factor value is determined by measuring a medium-sized time segment containing 100 short time-segments, hence determining the value of 100 pattern factors. The variation between the value of these pattern factors reflects the effect of artefacts. When the variation between the value of pattern factors is very small, the likelihood of an occurring artefact is very small.

To enable a rejection/acceptance-process, a threshold may be required for the pattern recognition at S250. For instance, the threshold may be defined such that the value of a pattern factor may not deviate more than 5% of the mean value as determined in the controlled experiment without artefacts. The threshold may be adapted based on a desired clinical required accuracy. If a particular pattern factor is above the threshold, the affiliated short time-segment is removed from the data set received from the consumer device. This removal may be limited to when there is a variation around the mean. When there is a trend to increasing deviation from the mean, the limitation may not be applied to involved short-time segments.

When more datasets become available based on measurements performed in the field, the mean value of the pattern factors may deviate from the mean value of the pattern factors of the controlled experiments without artefacts. In this case, the mean value of the pattern factors as determined in the field may be used, particularly when it is plausible that the chosen pattern factor acts as a fully random occurrence around the mean.

Further artefacts can express peculiar effects, for instance always occur in repeats of two. In the example of the peak-to-peak distance, a deviation above the threshold only occurs in two subsequent time-distances. This helps to improve the accuracy of removing pattern factors with too much deviation.

Additionally, other pattern factors may be used for the same dataset and may improve the specificity of detecting artefacts. As an example, pattern factors can be: (i) the speed-gradient within the pattern, (ii) the repeat frequency of a pattern, (iii) the change in average time distance between peaks in a pattern.

If no pattern is recognized at S250 (S250=No), at S255 the method of FIG. 2 stops at S255.

If a pattern is recognized at S250 (S250=Yes), at S260 the data is cleaned. The data may be cleaned at S260 by removing data segments containing too may artefacts. At S260, unavoidable artefacts present in the parameter data are filtered out to an acceptable level. For this a pattern recognition algorithm is employed using the characteristics of the patterns. From the gathered parameters over time, both physiological signals as well as the presence and concentration of chemical biomarkers, so called pattern factors are determined. Pattern factors can be regarded as aggregated data; for instance, measuring the average time between peaks of a pattern as collected in a relative short time duration. Subsequently, a comparison is made towards previous measured patterns and calculated pattern factors. This comparison will reveal a variation in the value of these factors. If these variations express a randomness-like behavior, they are attributed to artefacts and subsequently rejecting those data that contain too much random-like behavior according to a set threshold. If during the first period (week to a few months) of use random-like features occur above the set threshold, special action will be taken by the system: request to start again and pay attention to prevent artefacts and if this does not help the consumer device has to be regularly controlled on correct use in this first period by a healthcare professional, such as for example a nurse, a GP-assistant. Noteworthy is that in PPG and actigraphy sensors artefact cleaning is included in the internal signal processing and this can support the new system as well. However, PPG and actigraphy are a subset of the parameters that can be measured.

At S265, the cleaned data is stored for future reference. For example, cleaned data may be stored for processing at S270, S275, S280 and S285.

At S270, the method of FIG. 2 includes detecting deterioration by trend identification. Detecting deterioration may be valuable in a variety of contexts. For example, a patient discharged from a hospital may be followed at home during recovery to ensure deterioration in health is not occurring. During the development phase for the system 100, thresholds for measured parameter data and pattern factors may be determined. The thresholds may be applied as criteria for the level of randomness. The thresholds may take into account a constant or steady evolvement and may be used to identify and confirm trends. The trend information is used to detect the level of deterioration.

If the variations reveal a trend-like behavior, the variations may be attributed to a change in health status. If the trends constitute a relatively large deviation, this can be attributed to a diseased state of a person. Note that the increased occurrence of a random-like signal can also be a trend. Nevertheless, if the disorder gets worse the worsening may be noted as a trend. A modest deviation can be used as an early warning, and in turn utilized to invoke a preventive measure. Preventative measures may range from inducing a change in consumer lifestyle behavior up to alerting a crash team in the hospital in case a serious disorder is imminent.

During development, patterns as recorded from healthy persons and diseased persons can be used to identify those pattern factors that are most specific. For this, healthy persons and diseased persons are followed overtime. This includes pattern recognition by studying combined data from various sensors. For example, actigraphy can provide some context and reveal if SpO2 levels are different due to a different activity level. When a healthy person becomes ill, a precise comparison of the pattern factors relevant to a trend pattern change may be established.

A Fourier transform may be used as a filter to transform data as function of time to data as function of frequency, rendering a fingerprint in the frequency domain. For example, a constant pattern may show strong signals at certain frequencies. But also, when a disorder induces a trend towards a new status in the pattern, this new status may show strong signals at certain frequencies. Even during the change from one to another status, the signals will be still larger than from random occurring effects. Broadening of a signal may be observed during the switch from healthy to a diseased state. Since (semi)continuous measurements render a lot of data, the Fourier transform may be used to enhance precision. The signal variability of a pattern without artefacts in the frequency domain is typically limited due to having well defined features in the frequency domain. In the frequency domain, random occurring artefacts should only provide a signal of small value and spread over various frequencies. A constant pattern will be evidenced by strong signals at certain frequencies. When a disorder induces a trend towards a new status in the pattern, this new status may show strong signals at certain frequencies. By selecting the signals at particular frequencies where strong signals are detected, random data can be removed.

At S275, a check is made to confirm that the data are qualified in such a manner that they can be used for medical purposes, such as supporting data that are obtained by medical device(s) or such as supporting clinicians in obtaining a medical status of a person under question or such as independent data containing patterns that are a strong indication of deterioration. The detection of an upcoming disorder by the artificial intelligence engine at S245 may be compared with the trend identification at S270. If the results of S245 and S270 agree, the trend pattern factors are released for use in the medical practice by communicating these factors to the EMR 190 and/or a monitoring system 170, such as a patient monitor. The use of trend pattern factors at S270 may lead to an identification of an upcoming disorder and this will be compared with the identification of an upcoming disorder by the artificial intelligence engine applied at S245. When in accordance, the trend pattern factors and identification of upcoming disorder may be released for use in medical practice. The trend pattern factors and identification of upcoming disorder may be sent to the EMR 190 and/or monitoring system 170, such as a patient monitor. The artificial intelligence engine applied at S245 may be updated by use of (i) data of the person wearing the consumer device and by (ii) data as gathered anonymously for many persons. This will improve the specificity and can help in improving threshold of the trend pattern factors.

If the data qualification is insufficient for medical use (S275=No), at S280 the method of FIG. 2 stops. The data may be discarded once a determination is made that such data is inappropriate for storage in the EMR 190 or for processing, storage and/or display in the monitoring system 170.

If the data are qualified for medical use, at S285 the data is released to an electronic medical records system such as the EMR 190 in FIG. 1 and/or to a monitoring system 170, such as a patient monitor. The results of applying randomness criteria and trend criteria at S270 enable the use of a quality index of the data. For example, a quality index may be based on the percentage of data removed due to artefact reduction. A reliability threshold may be set to a maximum amount of removed data, when below the threshold the data are allowed to be used in a medical environment. If favorable, the data are released.

Data may be gathered and analyzed for a length of time such as a week or more before the data is used to identify an upcoming disorder and made available for release. Imposing a minimum study time may enable personalized reference values for a healthy state for a person, as compared to data from the consumer devices when the person becomes diseased. The confirmation of a health state may initially be deferred until adequate data is collected for the person. The question may arise as to how the absence of a disorder can be known during the first time-duration. This is carried out by a first analysis of patterns, comparing the patterns with normal values.

At S290, the data is stored, such as in the EMR 190 or monitoring system 170 in FIG. 1. The gateway computer 150 may release and classify the data for storage in the EMR 190 and/or a monitoring system 170, such as a patient monitor. Release to the EMR 190 may be oriented to establishing the condition of a patient. Release to a monitoring system 170 such as a patient monitor may be oriented to relative fast changing patterns, such as identifying an upcoming disorder affiliated to early warning. Due to the large amount of data from consumer devices, medical professionals may select to display an aggregated format of the data or may select to have the data from the nested applications limited to a summary form or another form of abbreviated data. By sending measured data and/or trend patterns to patient monitors, this information may be input to early warning scoring solutions. When measured data and/or trend patterns are sent to a patient monitor, this information can be used to set personalized alarms since a baseline is available from the measurements performed outside the hospital setting. Since different patients may have different baselines such as different heart rates in resting condition, more personalized alarms can lead to fewer false alarms in the hospital setting.

While the data generated by consumer devices is not regulated by MDR or IVDD, the data can still be used to induce enhanced surveillance of a particular patient as currently done by classical vital signs as measured by medical devices. Another use is to compare the data when the person was still healthy and then when diseased, particularly for persons where the vital signs as measured by medical devices are inconclusive on the basis of normal values.

When classification has been established, the data generated by consumer devices may also be corrected to enhance accuracy of recommendations for behavioral adjustments to treat a disorder. The data may also play a role in monitoring a patient at home; for instance, when a medical problem occurs after premature release, this problem may be picked up and an early warning is given to the patient at home or directly sent to a hospital system that alerts a doctor of the early warning or directly send to an application on a smart phone alerting a doctor of the early warning.

Although not described for FIG. 2, the system 100 may include a calibration protocol. The calibration protocol may check the correlation between values of a consumer device and the values of a medical device such values provided to a patient monitor system or sensed by a medical patch. The calibration protocol may be performed simultaneously, such as when a person is admitted to a hospital. The calibration protocol may induce a correction factor into the data as obtained by the consumer device. In some embodiments, when calibration is not performed for a consumer device for a predetermined time, data from the consumer device may be rejected until the calibration is performed.

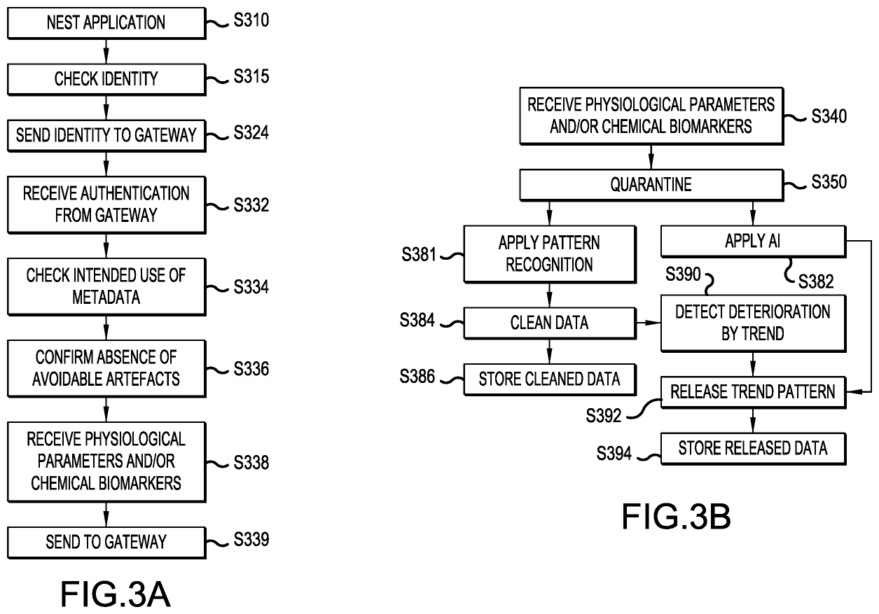

FIG. 3A illustrates another method for selectively repurposing consumer device data, in accordance with a representative embodiment.

The method of FIG. 3A may be performed by and/or on one of the first consumer device 101 or the second consumer device 102 in FIG. 1, and is relatively more detailed than the corresponding functionality briefly shown in and described with respect to FIG. 2.

At S310, an application is nested. The application may be nested when the first consumer device 101 or the second consumer device 102 are built, or may be subsequently downloaded from the internet to or otherwise stored as an update on the first consumer device 101 or the second consumer device 102.

At S315, an identity is checked. The identity may be affirmatively checked by the nested application asking a user to login with a username and password, passively by the nested application monitoring the user as the user interacts with the consumer device, or both actively and passively. For example, the nested application may authenticate the measured data with the help of the gateway computer 150 in that the gateway computer 150 provides identification parameters such as name, address, gender, and/or age as stored in the EMR 190.

At S324, data is sent to a gateway. The data sent to the gateway at S324 may be a purported identity of a user. At S332, authentication is received from the gateway, such as when the gateway confirms that the user is being monitored by a system that includes the gateway.

At S334, the intended use of metadata is checked. The intended use consistent with the teachings herein should be for an analysis related to health/medical purposes, and if the intended use is not consistent with these purposes, the process may be stopped after S334.

At S336, the absence of avoidable artefacts is confirmed. The consumer device may analyze data being collected and identify avoidable artefacts. The absence of avoidable artefacts may be an absolute requirement or a relative requirement. For example, the consumer device may compare the volume of avoidable artefacts to a threshold. The threshold may vary based on context, such as the type of consumer device, the reasons for which the nested application has been installed on the consumer device, and/or demographic or medical characteristics of the consumer.

At S338, physiological parameters and/or chemical biomarkers are received. The physiological parameters and/or chemical biomarkers may be directly sensed by the consumer device, or may be based on or derivable from data received by the consumer device.

At S339, data of the physiological parameters and/or chemical biomarkers is sent to the gateway. The data sent to the gateway may include data sensed by, received by, or derived by the consumer device, as well as metadata for the data sent to the gateway.

As should be clear, the method of FIG. 3A assumes that the first consumer device 101 and/or the second consumer device 102 is configured to connect to the gateway computer 150 in FIG. 1 over the internet. The first consumer device 101 and/or the second consumer device 102 are also configured to check identities of one or more users, either actively such as by checking a login via a username and password or personal identification number, or passively by checking for a characteristic of a user. For example, if the first consumer device 101 is a smart scale, the first consumer device 101 may be instructed to send weights for one user but not for other users, and may recognize the one user based on the weight of the one user falling within a range that is not expected for other users of the smart scale. Identification parameters may include name, address, gender, age, password, personal identification number and/or more.

FIG. 3B illustrates another method for selectively repurposing consumer device data, in accordance with a representative embodiment.

The method of FIG. 3B may be performed by the gateway computer 150 in FIG. 1, but is otherwise somewhat duplicative of the method in FIG. 2 from S225 to S290.

At S340, data of physiological parameters and/or chemical biomarkers are received. The data is received from a consumer device and comprises data received by or sensed by the consumer device, or metadata generated based on data received by or sensed by the consumer device. The physiological parameters and/or chemical biomarkers may be provided as data from direct measurements reflective of physiology and/or chemical biomarkers, or may be provided based on analysis of operations of the consumer device. For example, analysis by the consumer device may comprise determinations of a volume or speed of speech by a user on a cell phone, and whether the volume or speed of speech is above or below a threshold.

At S350, the data received at S340 is quarantined. The data may be quarantined locally by the gateway computer 150 while processing is performed on the data.

At S381, pattern recognition is applied. The gateway computer 150 may execute algorithms to (i) classify time-segments of the data from the consumer device, and (ii) clean the data at S384 to remove time-segments with a high level of artefacts over a threshold. The data may be cleaned by comparing data within a data set of interest. The data set may be divided up into segments, and outlier values for segments may be compared to an average across segments. The average may be calculated per segment. Subsequently the variation over all segments of this average pattern factor may be determined, for instance by determining the standard deviation, using a bin method. The bin method counts the number of pattern factors in a particular time range for the bin. When the standard deviation is too broad and/or the plot deviates from a gaussian curve, the gateway computer 150 may conclude that the data includes substantial randomness. The data set may be accepted or rejected based on threshold values for the standard deviation and deviation from the gaussian curve shape. At S386, after S384, the cleaned data is stored.

At S390, also after S384, deterioration is detected by trend. The gateway computer150 may determine trends and affiliated disorders by an algorithm determining trend pattern factors and via thresholds which provide a trend identification representing the detection of a patient's deterioration.

At S382, artificial intelligence is applied in parallel with S381. An artificial intelligence engine may be applied to detect deterioration in a patient. Deterioration may be detected by comparing the data most recently received from the consumer device to previous and comparable data received from the consumer device or other consumer devices of the consumer.

At S392, the trend pattern is released. If the artificial intelligence results and the trend detection are in accordance, the trend pattern factor and detection of upcoming disorder is released.

At S394, the released data is stored. The released data may be released to and stored by an EMR 190, and/or may be released to a patient monitor such as the monitoring system 170.

The cleaning as part of the pattern recognition at S381 and the detection of deterioration at S390 may also benefit from other parameters which may be sensed and which provide data patterns as well. Examples of other parameters include SpO2 sensor data, capnography, sweat excretion. In SpO2 sensing, the signal is relatively small with respect to the noise background and the signal is filtered by the fact that the volume of blood passing through the finger is increased during the heartbeats. The difference in the signal between increased blood flow and non-increased blood flow is less determined by the noise. As a result, the pattern in heart beats may be derived from the SpO2 signal. Moreover, in capnography, the CO2 concentration is determined in the inhaled and exhaled air, and the signal also includes the respiratory rate. As a result, the respiratory rate may be derived from the determinations of CO2 readings, consistent with the teachings herein. Sweat excretion of an individual sweat gland also may provide a pattern. Sweat rate per gland may be measured and the number of active sweat glands may be determined. A typical behavior of an active sweat gland is to excrete in a cyclic manner, for example a cycle consists out of excreting for 30 seconds and then stops excreting for about 150 seconds. The sweat cycles may vary from person to person. When a human body needs to increase the sweating, the amount of produced sweat per gland is increased and the number of active sweat gland per skin surface area is increased, changing the sweat pattern. A change in sweat pattern (in time and in sweat rate) of a patient may indicate a change in physiology including an upcoming disorder. For example, a change in sweat pattern may serve as a proxy for core body temperature, and with proper modulation may be used to sense an upcoming disorder. Sweat sensing may also be used to determine concentration profiles of a particular chemical biomarker in sweat, including, for example, glucose, lactate, melatonin, cortisol and electrolytes. As an example, glucose may be measured to determine the pattern during the day and night and, when diseased, will have too low or too high values. Glucose has a clear pattern depending on food intake. Lactate has a relatively constant pattern when a person is resting, but when diseased has a run-away trend with an exponential increase. Melatonin concentration follows a day-night rhythm except when being disturbed by a sleep disorder. Cortisol is a so-called stress hormone and can play an important role in determining Delirium and also has a rhythm. Electrolytes have a direct relation to dehydration. Chemical biomarkers which may be measured by a consumer device in the future may also contribute to the detection of deterioration as other parameters at 390, after appropriate cleaning at S381. A variety of other parameters may therefore add to the analysis at the gateway computer 150 and in the system 100 as a whole.

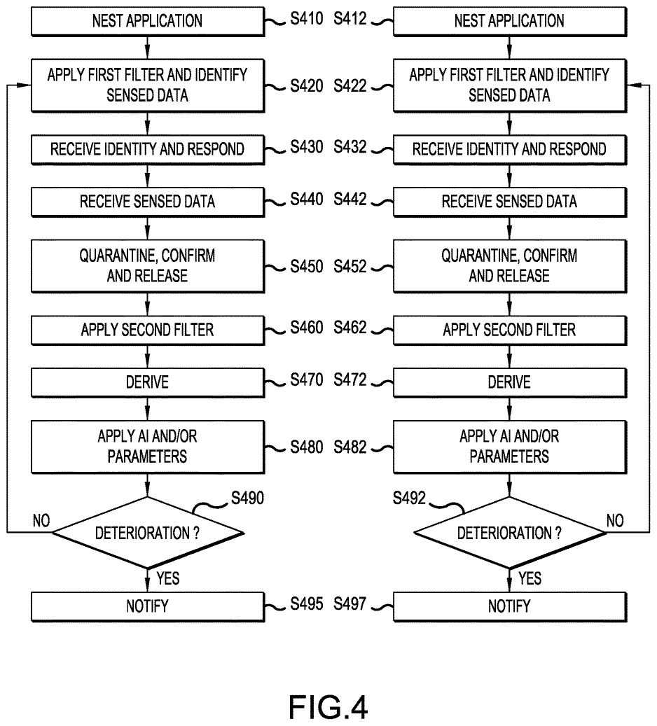

Additionally, the release of trend patterns at S394 by the gateway computer 150 may also be accompanied by a quality index. When the trend pattern factors and/or aggregated analysis are released they may be accompanied by a quality index with multiple potential levels such as low, medium and high. A low level may correspond to data which should not be used, and which may not be released to the monitoring system 170 and/or the EMR 190. A medium level may correspond to data which is released to the monitoring system 170 and/or the EMR 190, but which should be used to support data as obtained by medical devices. For example, a medium level may be assigned to data sets that were recorded when the person was not ill and which may be used as personalized normal values. When the data of the consumer device are in contradiction with the data of the medical device(s), the medium level may serve as the basis for advising remeasurement of the medical data and medical handling will only be based on the data of the medical data from the medical device(s). A high level may be assigned to data considered to be of such high value that it can be properly classified as medical data and released for sending to the monitoring system 170 and the EMR 190. For example, the high value may be assigned to data sets that were recorded when the person was not ill and which may be used as personalized normal values. The high value data may also be used in combination with the data as obtained by medical devices to define medical handling. The data from consumer devices may be classified by a quality index and useful in the medical context, such as when the data from the consumer devices shows the patterns of a person in healthy states and in diseased states and over a long time compared to measurements in a medical setting. Moreover, measurement with a sensor with relatively large variability may still determine the mean of a parameter accurately due to the use of a large dataset, and many consumer devices may be considered sensors that provide data sets with potentially large variability. The quality index may be built by comparing percentage of the data removed due to randomness with two thresholds. When the percentage is above the highest threshold, the data may be classified as low and rejected for further use. When in between the two thresholds, the data may be classified as medium. When below the lowest threshold, the data may be classified as high. The thresholds may be quantified during development and/or a clinical study. The data classification may be used by the gateway computer 150 to determine the use of data from consumer devices, and the consumer devices may be prevented from directly communicating with the monitoring system 170 or the EMR 190. FIG. 4 illustrates another method for selectively repurposing consumer device data, in accordance with a representative embodiment.

The method of FIG. 4 is performed by two consumer devices, such as the first consumer device 101 and the second consumer device 102. A first part of FIG. 4 is performed on the left and a second part of FIG. 4 is performed on the right, though the two parts may be performed simultaneously or asynchronously and at different times. Relative to FIG. 2, the method of FIG. 4 also highlights the application of multiple filters between the consumer devices and the gateway computer 150. Two or more checks may be imposed in processes described herein to confirm the applicability of data for medical purposes as well as to confirm the identity of a user of the consumer device by which the data is being sent to the gateway computer 150.

At S410, an application is nested on a consumer device such as the first consumer device 101. At S412, an application is nested on a consumer device such as the second consumer device 102. The nested applications may be the same application or different applications.

At S420, the first consumer device applies a first filter, and identifies sensed data. At S422, the second consumer device applies a first filter and identifies sensed data. The sensed data may be the data sensed or to be sensed by components of the first consumer device and the second consumer device, and which is left after the first filters are applied. The first filter may simply detect unavoidable artefacts known to and recognized by the first consumer device and the second consumer device, such as data segments occurring when the first consumer device and second consumer device are dropped.

At S430, the first consumer device receives an identification and responds. At S432, the second consumer device receives an identification and responds. The identification may be received from the gateway computer 150, and may comprise identification information corresponding to a person being monitored by the nested applications. The identification may be compared with the current user(s) of the first consumer device and the second consumer device, and the process of FIG. 4 may be stopped if the current user(s) do not match the identification information corresponding to the person being monitored by the nested applications. The response may include notifying the gateway computer 150 whether the identification matches or not, and thus whether the remainder of the method in FIG. 4 will be performed or not.

At S440, the first consumer device receives sensed data. At S442, the second consumer device receives sensed data.

At S450, the data from the first consumer device is quarantined, confirmed and released. At S452, the data from the second consumer device is quarantined, confirmed and released. The quarantining at S450 and S452 is performed by the gateway computer 150 after the data is sent from the first consumer device and the second consumer device.

At S460, a second filter is applied to the data from the first consumer device. At S462, a second filter is applied to the data from the second consumer device. The second filter is described above with respect to S150 and S260 with respect to FIG. 2. The second filters may be applied to remove unavoidable artefacts present in the parameter data so as to achieve an acceptable level.

At S470, a pattern is derived and characterized from the data from the first consumer device. At S472, a pattern is derived and characterized from the data from the second consumer device. The pattern may reflect physiological characteristics of the consumer.

At S480 artificial intelligence and/or parameters is/are applied to the data from the first consumer device. At S482, artificial intelligence and/or parameters is/are applied to the data from the second consumer device.

At S490, a determination is made as to whether the data from the first consumer device indicates deterioration. At S492, a determination is made as to whether the data from the second consumer device indicates deterioration.

If there is no deterioration at S490, the process on the left for the first consumer device returns to S420. If there is no deterioration at S492, the process on the right for the second consumer device returns to S422.

If deterioration is detected at S490, a notification is made at S495. If deterioration is detected at S492, a notification is made at S497.

While the method of FIG. 4 includes two flows for two consumer devices, it should be clear that a single person may have one or more consumer devices sending data to the gateway computer. Moreover, from the viewpoint of the gateway computer, data may be received from numerous consumer devices owned by numerous different people. For example, the gateway computer may be implemented in the cloud at a data center, and may receive at any one time data from numerous

FIG. 5 illustrates an example of variable interbeat time duration for selectively repurposing consumer device data, in accordance with a representative embodiment.

FIG. 5 shows an example of variable interbeat time duration on a user interface 581 used for an internal check to authenticate data at the gateway computer 150 at S240. For example, a pattern factor is the time between heartbeats. Subsequently the number of pattern factors as function of time can be plotted using bins and this plot can be compared between different datasets. The gateway computer 150 may check a current reading of interbeat time duration against a pattern derivable from previous readings, and this type of check may be required before data is released from quarantine. For example, confirming that data reflects a physiological event may be performed based on a pattern detected on data previously received from the consumer device of the subject.

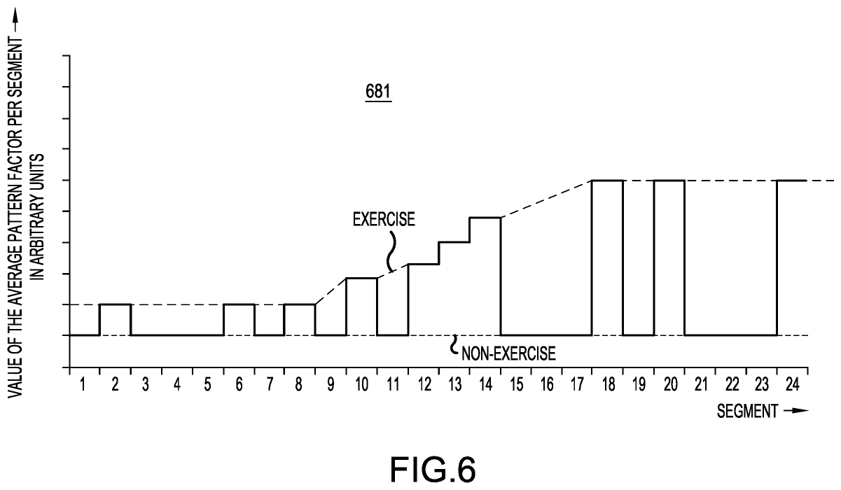

FIG. 6 illustrates average pattern factor per time segment as a function of the number of segments measured in a chronologic sequence for selectively repurposing consumer device data, in accordance with a representative embodiment.

In FIG. 6, an average pattern factor per time segment is shown on a user interface 681 as a function of the number of segments measured in chronologic sequence. In this case, the upcoming disorder can be measured even with a high level of random occurrences. In this case, the upcoming disorder is only revealed when there is a certain level of exercise due to walking the stairs. The rough dashed curve across the top of readings indicates the level in case of exercise and the fine dashed horizontal curve at the bottom indicates the level in case of no exercise. Staying with the example of heart rate, the pattern factors “peak to peak time duration” may be determined per segment and the average may be calculated per segment. Subsequently, the average value of the pattern factor per segment is plotted of the segments as they occur over time. When the value of the “average pattern factor per segment” in this plot shows an increase or decrease, the change may indicate a change in the pattern that can be attributed to a change in health status and when the change is large it may reveal an upcoming disorder. Typically, an upcoming disorder occurs over a longer time period than a random artefact. In some instances, a change in health status may only be revealed when the person is in a certain state, for instance during walking stairs. In this case, evaluating the “average pattern factor per segment” plot as in FIG. 6 will reveal two levels of this pattern factor that occur in a seemingly random fashion since walking on the stairs is relatively random. Nevertheless, the level of this pattern factor related to walking the stairs will show an upward trend as in the example of FIG. 6, and the gateway computer 150 may conclude that a change in health status is occurring.

FIG. 7 illustrates a computer system, on which a method for selectively repurposing consumer device data is implemented, in accordance with another representative embodiment.

Referring to FIG. 7, the computer system 700 includes a set of software instructions that can be executed to cause the computer system 700 to perform any of the methods or computer-based functions disclosed herein. The computer system 700 may operate as a standalone device or may be connected, for example, using a network 701, to other computer systems or peripheral devices. In embodiments, a computer system 700 performs logical processing based on digital signals received via an analog-to-digital converter.

In a networked deployment, the computer system 700 operates in the capacity of a server or as a client user computer in a server-client user network environment, or as a peer computer system in a peer-to-peer (or distributed) network environment. The computer system 700 can also be implemented as or incorporated into various devices, such as the gateway computer 150, a computer of the monitoring system 170, a computer specific to the EMR 190, a workstation that includes a controller, a stationary computer, a mobile computer, a personal computer (PC), a laptop computer, a tablet computer, or any other machine capable of executing a set of software instructions (sequential or otherwise) that specify actions to be taken by that machine. The computer system 700 can be incorporated as or in a device that in turn is in an integrated system that includes additional devices. In an embodiment, the computer system 700 can be implemented using electronic devices that provide voice, video or data communication. Further, while the computer system 700 is illustrated in the singular, the term “system” shall also be taken to include any collection of systems or sub-systems that individually or jointly execute a set, or multiple sets, of software instructions to perform one or more computer functions.

As illustrated in FIG. 7, the computer system 700 includes a processor 710. The processor 710 may be considered a representative example of a processor of a controller and executes instructions to implement some or all aspects of methods and processes described herein. The processor 710 is tangible and non-transitory. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The processor 710 is an article of manufacture and/or a machine component. The processor 710 is configured to execute software instructions to perform functions as described in the various embodiments herein. The processor 710 may be a general-purpose processor or may be part of an application specific integrated circuit (ASIC). The processor 710 may also be a microprocessor, a microcomputer, a processor chip, a controller, a microcontroller, a digital signal processor (DSP), a state machine, or a programmable logic device. The processor 710 may also be a logical circuit, including a programmable gate array (PGA), such as a field programmable gate array (FPGA), or another type of circuit that includes discrete gate and/or transistor logic. The processor 710 may be a central processing unit (CPU), a graphics processing unit (GPU), or both. Additionally, any processor described herein may include multiple processors, parallel processors, or both. Multiple processors may be included in, or coupled to, a single device or multiple devices.

The term “processor” as used herein encompasses an electronic component able to execute a program or machine executable instruction. References to a computing device comprising “a processor” should be interpreted to include more than one processor or processing core, as in a multi-core processor. A processor may also refer to a collection of processors within a single computer system or distributed among multiple computer systems. The term computing device should also be interpreted to include a collection or network of computing devices each including a processor or processors. Programs have software instructions performed by one or multiple processors that may be within the same computing device or which may be distributed across multiple computing devices.

The computer system 700 further includes a main memory 720 and a static memory 730, where memories in the computer system 700 communicate with each other and the processor 710 via a bus 708. Either or both of the main memory 720 and the static memory 730 may be considered representative examples of a memory of a controller, and store instructions used to implement some or all aspects of methods and processes described herein. Memories described herein are tangible storage mediums for storing data and executable software instructions and are non-transitory during the time software instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The main memory 720 and the static memory 730 are articles of manufacture and/or machine components. The main memory 720 and the static memory 730 are computer-readable mediums from which data and executable software instructions can be read by a computer (e.g., the processor 710). Each of the main memory 720 and the static memory 730 may be implemented as one or more of random access memory (RAM), read only memory (ROM), flash memory, electrically programmable read only memory (EPROM), electrically erasable programmable read-only memory (EEPROM), registers, a hard disk, a removable disk, tape, compact disk read only memory (CD-ROM), digital versatile disk (DVD), solid state drive (SSD), floppy disk, blu-ray disk, or any other form of storage medium known in the art. The memories may be volatile or non-volatile, secure and/or encrypted, unsecure and/or unencrypted.

“Memory” is an example of a computer-readable storage medium. Computer memory is any memory which is directly accessible to a processor. Examples of computer memory include, but are not limited to RAM memory, registers, and register files. References to “computer memory” or “memory” should be interpreted as possibly being multiple memories. The memory may for instance be multiple memories within the same computer system. The memory may also be multiple memories distributed amongst multiple computer systems or computing devices.

As shown, the computer system 700 further includes a video display unit 750, such as a liquid crystal display (LCD), an organic light emitting diode (OLED), a flat panel display, a solid-state display, or a cathode ray tube (CRT), for example. Additionally, the computer system 700 includes an input device 760, such as a keyboard/virtual keyboard or touch-sensitive input screen or speech input with speech recognition, and a cursor control device 770, such as a mouse or touch-sensitive input screen or pad. The computer system 700 also optionally includes a disk drive unit 780, a signal generation device 790, such as a speaker or remote control, and/or a network interface device 740.

In an embodiment, as depicted in FIG. 7, the disk drive unit 780 includes a computer-readable medium 782 in which one or more sets of software instructions 784 (software) are embedded. The sets of software instructions 784 are read from the computer-readable medium 782 to be executed by the processor 710. Further, the software instructions 784, when executed by the processor 710, perform one or more steps of the methods and processes as described herein. In an embodiment, the software instructions 784 reside all or in part within the main memory 720, the static memory 730 and/or the processor 710 during execution by the computer system 700. Further, the computer-readable medium 782 may include software instructions 784 or receive and execute software instructions 784 responsive to a propagated signal, so that a device connected to a network 701 communicates voice, video or data over the network 701. The software instructions 784 may be transmitted or received over the network 701 via the network interface device 740. The network interface device 740 may include an analog-to-digital converter that converts analog signals received over the network 701 into digital signals.

In an embodiment, dedicated hardware implementations, such as application-specific integrated circuits (ASICs), field programmable gate arrays (FPGAs), programmable logic arrays and other hardware components, are constructed to implement one or more of the methods described herein. One or more embodiments described herein may implement functions using two or more specific interconnected hardware modules or devices with related control and data signals that can be communicated between and through the modules. Accordingly, the present disclosure encompasses software, firmware, and hardware implementations. Nothing in the present application should be interpreted as being implemented or implementable solely with software and not hardware such as a tangible non-transitory processor and/or memory.

In accordance with various embodiments of the present disclosure, the methods described herein may be implemented using a hardware computer system that executes software programs. Further, in an exemplary, non-limited embodiment, implementations can include distributed processing, component/object distributed processing, and parallel processing. Virtual computer system processing may implement one or more of the methods or functionalities as described herein, and a processor described herein may be used to support a virtual processing environment.

Accordingly, selectively repurposing consumer device data enables upgrading of the quality of data collected by consumer devices to data that can be used in the medical domain.

Although selectively repurposing consumer device data has been described with reference to several exemplary embodiments, it is understood that the words that have been used are words of description and illustration, rather than words of limitation. Changes may be made within the purview of the appended claims, as presently stated and as amended, without departing from the scope and spirit of selectively repurposing consumer device data in its aspects. Although selectively repurposing consumer device data has been described with reference to particular means, materials and embodiments, selectively repurposing consumer device data is not intended to be limited to the particulars disclosed; rather selectively repurposing consumer device data extends to all functionally equivalent structures, methods, and uses such as are within the scope of the appended claims.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

1. A system for deriving medical characteristics from data, comprising:
a gateway computer comprising a memory that stores instructions and a processor that executes the instructions, wherein, when executed by the processor, the instructions cause the gateway computer to:
receive first data from a first consumer device;
filter the first data for quality based on a first pattern of the first data and to eliminate artefacts to produce first filtered data;
derive, from the first filtered data, a first physiological parameter;
receive, from a second consumer device, second data;
filter the second data for quality based on a second pattern of the second data and to eliminate artefacts to produce second filtered data; and
derive, from the second filtered data, a second physiological parameter.

2. The system of claim 1, wherein the gateway computer receives the first data via a first application nested in the first consumer device, and the second data via a second application nested in the second consumer device.

3. The system of claim 2, wherein, when executed by the processor, the instructions further cause the gateway computer to:
receive identification information of a subject from the first application, and confirm that the gateway computer is authorized to receive the first data for the subject;
respond to the first application to confirm that the gateway computer is authorized to receive the first data for the subject before receiving the first data from the first consumer device;
receive identification information of the subject from the second application, and confirm that the gateway computer is authorized to receive the second data for the subject; and
respond to the second application to confirm that the gateway computer is authorized to receive the second data for the subject before receiving the second data from the second consumer device.

4. The system of claim 3, wherein the first application nested in the first consumer device identifies the first data by filtering data sensed by the first consumer device, and wherein the second application nested in the second consumer device identifies the second data by filtering data sensed by the second consumer device.

5. The system of claim 3, wherein the gateway computer receives the first data and the second data in a format required by the gateway computer, wherein the format includes a source identification field to identify the first consumer device or the second consumer device and a source identification field to identify the subject.

6. The system of claim 2, wherein, when executed by the processor, the instructions cause the gateway computer further to:
quarantine the first data and the second data while filtering and eliminating artefacts; and
confirming that the first data reflects the first physiological parameter and confirming that the second data reflects the second physiological parameter, and
releasing the first data and the second data based on confirming that the first data reflects the first physiological parameter and based on confirming that the second data reflects the second physiological parameter.

7. The system of claim 6,
wherein the confirming that the first data reflects the first physiological parameter is performed based on a pattern detected on data previously received from the subject, and
wherein the confirming that the second data reflects the second physiological parameter is performed based on a pattern detected on data previously received from the subject.

8. The system of claim 3, wherein, when executed by the processor, the instructions cause the gateway computer further to:
detect deterioration in health of the subject based on at least one of the first data or the second data.

9. The system of claim 8, wherein, when executed by the processor, the instructions cause the gateway computer further to:
apply a trained artificial intelligence model to the first data and the second data to detect the deterioration in health of the subject.

10. The system of claim 8, wherein, when executed by the processor, the instructions cause the gateway computer further to:
apply parameters customized for the subject to detect the deterioration in health of the subject.

11. A method for deriving medical characteristics from data, comprising:
receiving, at a gateway computer comprising a memory that stores instructions and a processor that executes the instructions, from a first consumer device, first data;
filtering, by the gateway computer, the first data for quality based on a first pattern of the first data and to eliminate artefacts to produce first filtered data;
deriving, by the gateway computer, from the first filtered data, a first physiological parameter;
receiving, by the gateway computer, from a second consumer device, second data;
filtering, by the gateway computer, the second data for quality based on a second pattern of the second data and to eliminate artefacts to produce second filtered data; and
deriving, by the gateway computer, from the second filtered data, a second physiological parameter.

12. The method of claim 11, further comprising:
receiving the first data via a first application nested in the first consumer device, and the second data via a second application nested in the second consumer device.

13. The method of claim 12, further comprising:
receiving identification information of a subject from the first application, and confirming that the gateway computer is authorized to receive the first data for the subject;
responding to the first application to confirm that the gateway computer is authorized to receive the first data for the subject before receiving the first data from the first consumer device;
receiving identification information of the subject from the second application, and confirming that the gateway computer is authorized to receive the second data for the subject; and
responding to the second application to confirm that the gateway computer is authorized to receive the second data for the subject before receiving the second data from the second consumer device.

14. The method of claim 13, further comprising:
identifying, by the first application nested in the first consumer device, the first data by filtering data sensed by the first consumer device, and
identifying, by the second application nested in the second consumer device, the second data by filtering data sensed by the second consumer device.

15. The method of claim 13,
wherein the first data and the second data are received in a required format, wherein the required format includes a source identification field to identify the first consumer device or the second consumer device and a source identification field to identify the subject.

16. The method of claim 12, wherein, when executed by the processor, the instructions cause the gateway computer further to:
quarantine the first data and the second data while filtering and eliminating artefacts; and
confirming that the first data reflects the first physiological parameter and confirming that the second data reflects the second physiological parameter, and
releasing the first data and the second data based on confirming that the first data reflects the first physiological parameter and based on confirming that the second data reflects the second physiological parameter.

17. The method of claim 16,
wherein confirming that the first data reflects the first physiological parameter is performed based on a pattern detected on data previously received from the subject, and
wherein confirming that the second data reflects the second physiological parameter is performed based on data previously received from the subject.

18. The method of claim 13, further comprising:
detecting deterioration in health of the subject based on at least one of the first data or the second data.

19. The method of claim 18, further comprising:
applying a trained artificial intelligence model to the first data and the second data to detect the deterioration in health of the subject.

20. The method of claim 18, further comprising:
applying parameters customized for the subject to detect deterioration in the health of the subject.
